SEQUENCE LISTING (12 sequences)

SEQ ID NO: 1 - DNA, 20 bp, Artificial Sequence
  Synthetic Primer
gggcatgaaccatgagaagt

SEQ ID NO: 2 - DNA, 20 bp, Artificial Sequence
  Synthetic primer
gtcttctgggtggcagtgat

SEQ ID NO: 3 - DNA, 20 bp, Artificial Sequence
  Synthetic primer
atttctcctggtgctgctgt

SEQ ID NO: 4 - DNA, 20 bp, Artificial Sequence
  Synthetic primer
ggaactccacaactgccaat

SEQ ID NO: 5 - DNA, 20 bp, Artificial Sequence
  Synthetic primer
cgagctgtggatgacttcaa

SEQ ID NO: 6 - DNA, 20 bp, Artificial Sequence
  Synthetic primer
tccttcactcggaacctcac

SEQ ID NO: 7 - DNA, 22 bp, Artificial Sequence
  Synthetic primer
cctcagggtaccactacggagt

SEQ ID NO: 8 - DNA, 18 bp, Artificial Sequence
  Synthetic primer
gccgaatagttcgccgaa

SEQ ID NO: 9 - DNA, 20 bp, Artificial Sequence
  Synthetic primer
tggagctcgatgacagtgac

SEQ ID NO: 10 - DNA, 20 bp, Artificial Sequence
  Synthetic primer
tgtcctggatggcttctacc

SEQ ID NO: 11 - DNA, 26 bp, Artificial Sequence
  Synthetic primer
gaagaactagagaaattggtagcaag

SEQ ID NO: 12 - DNA, 23 bp, Artificial Sequence
  Synthetic primer
ttcagctcatcactcagaatcag

(19) United States
(12) Patent Application Publication
(10) Pub. No.: US 2010/0173995 A1
(43) Pub. Date: Jul. 8, 2010

(54) Use of PPAR-alpha activator as inflammatory skin disease-treating agent and method for treating skin diseases using the same

(75) Inventors: Byeong-Deog Park, Cheongju (KR); Hyung-Sub Gwak, Dajeon (KR); Se-Kyoo Jeong, Dajeon (KR); Jong-Hwan Bae, Dajeon (KR)

Correspondence Address: Thomas M. Galgano, 20 W. Park Avenue, Suite 204, Long Beach, NY, 11561, US

(73) Assignee: Neopharm Co., Ltd.

(21) Appl. No.: 12/319,324

(22) Filed: Jan. 6, 2009

(51) Int. Cl. A61K 31/164; C07C 233/31; A61P 17/10

(52) U.S. Cl. 514/625; 564/199

(57) ABSTRACT

Disclosed herein is the use of a non-natural compound represented by the following formula 1 as an activator of the peroxisome proliferator activated receptor-α (PPAR-α) which shows an anti-inflammatory effect on inflammatory skin diseases. The PPAR-α activator increases the expression of PPAR-α in the skin to enhance various physiological effects of PPAR-α, that is, the anti-inflammatory effect caused by inhibiting the activity of transcription factors such as the nuclear factor κK (NF-κK) that induces inflammatory reactions, the effect of improving the epidermal permeability barrier function of the skin, and the effect of promoting the terminal differentiation of epidermal keratinocytes. Thus, the PPAR-α activator is useful for the treatment, alleviation or amelioration of skin diseases such as acne, seborrheic dermatitis, viral skin diseases, urticaria, pruritus, viral infectious diseases and contact dermatitis.

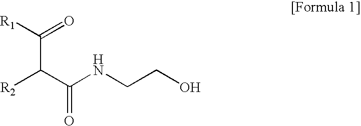

wherein R₁ and R₂ are the same or different and each represents a linear or branched C₁₋₂₂ alkyl group, a phenyl group or a benzene group.

### BACKGROUND OF THE INVENTION

1. Field of the Invention

The present invention relates to the use of a peroxisome proliferator activated receptor-α (PPAR-α) activator as an agent for treating skin diseases and a method for treating skin diseases using the same, and more particularly to the use of a compound represented by the following formula 1 as an activator for increasing the epidermal expression of PPAR-α, a method for activating epidermal PPAR-α, and a method of treating skin diseases using the aforementioned PPAR-α activator, in which the PPAR-α activator increases the expression of PPAR-α in the skin to enhance various physiological effects of PPAR-α, that is, the anti-inflammatory effect caused by inhibiting the activity of transcription factors such as the nuclear factor κK (NF-κK) that induces inflammatory reactions, the effect of improving the epidermal barrier function of the skin, and the effect of promoting the terminal differentiation of epidermal keratinocytes, and thus the PPAR-α activator can show the effects of treating and alleviating skin diseases such as acne, seborrheic dermatitis, viral skin diseases, urticaria, pruritus, viral infectious diseases and contact dermatitis.

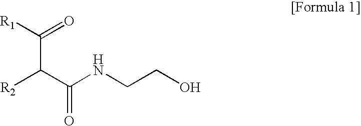

wherein R₁ and R₂ are the same or different and each represents a linear or branched C₁₋₂₂ alkyl group, a phenyl group or a benzene group.

2. Description of the Prior Art

The peroxisome proliferator activated receptor-α (PPAR) is a nuclear receptor that belongs to a superfamily of transcription factors. This superfamily includes steroids, retinoids and thyroid hormone receptors. Peroxisomes are intracellular organelles that are involved in oxidative action, are abundantly present in the liver and the kidneys and are also present in the skin. Peroxisome proliferators are compounds capable of increasing the number of peroxisomes and include fat and fatty acid, and fibrate and prostaglandin as hyperlipidemia-treating agents (Michalik et al, Pharmacol Rev 2006; 58: 726-741).

Three PPAR subtypes, that is, PPAR-α, PPAR-β/δ and PPAR-γ, were found in humans and rodents, and they are encoded by different genes and show different tissue distributions. Particularly, it is known that PPAR-α is expressed mainly in the liver and the skin, is involved in the oxidation of fatty acids or the neutralization of toxic substances and is associated with inflammatory reactions (Glass et al., Nat Rev Immunol 2006; 6: 44-55).

Also, PPAR-α has an anti-inflammatory effect, due to its inhibitory activity on transcription factors, such as the nuclear factor κK (NF-κK) that induces inflammatory reactions (Lefebvre et al., J Clin Invest 2006; 116: 571-580). Generally, PPARs function as sensors and regulators of inflammatory responses through their ability to be activated by locally generated eicosanoids (Moraes et al., Pharmacol Ther 2006; 110: 371-385). Such anti-inflammatory effect was proved from the fact that ear swelling was increased by leukotriene B4 in mice whose PPAR expressions has been removed through genetic manipulation (Devchand et al., Nature 1996; 384: 39-43). In addition, it was reported that PPAR-α has the effect of improving the epidermal permeability barrier function (Michalik et al., Biochim Biophys Acta 2007; 1771: 997-998) and the effect of promoting the terminal differentiation of epidermal keratinocytes (Komuves et al., J Invest Dermatol 2000; 115: 353-360), and thus it has the effects of alleviating various inflammatory skin diseases such as acne, seborrheic dermatitis, viral skin diseases, urticaria, pruritus, viral infectious diseases and contact dermatitis (Staumont-Salle et al., J Allergy Clin Immunol 2008; 121: 962-968). Accordingly, it is expected that inflammatory reactions can be inhibited or alleviated by using topical preparations containing a PPAR-α activator, which activates the epidermal PPAR-α.

### SUMMARY OF THE INVENTION

Accordingly, it is a first object of the present invention to provide the use of a PPAR-α activator as a therapeutic agent against skin diseases such as acne, seborrheic dermatitis, viral skin diseases, urticaria, pruritus, viral infectious diseases and contact dermatitis, in which the PPAR-α activator promotes the expression of PPAR-α to enable the physiological effects of PPAR-α to be effectively used.

A second object of the present invention is to provide a method of using a PPAR-α activator to prepare a topical composition for treating skin diseases such as acne, seborrheic dermatitis, viral skin diseases, urticaria, pruritus, viral infectious diseases and contact dermatitis.

A third object of the present invention is to provide a method for activating PPAR-α.

A fourth object of the present invention is to provide a method of using a PPAR-α activator to treat patients suffering from skin diseases such as acne, seborrheic dermatitis, viral skin diseases, urticaria, pruritus, viral infectious diseases and contact dermatitis.

The first object of the present invention may be effectively accomplished by a PPAR-α activator represented by the following formula 1:

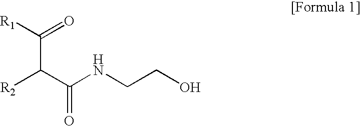

wherein R₁ and R₂ are the same or different and each represents a linear or branched C₁₋₂₂ alkyl group, a phenyl group or a benzene group.

The second object of the present invention may be accomplished by a method of using the PPAR-α activator represented by the formula 1 to prepare a topical composition for treating skin diseases, including acne, seborrheic dermatitis, viral skin diseases, urticaria, pruritus, viral infectious diseases and contact dermatitis, the PPAR-α activator being used in an amount of 0.1-15.0 wt % based on the total weight of the composition.

The third object of the present invention may be accomplished by a method comprising applying the PPAR-α activator represented by the formula 1 to the skin of patients suffering from skin diseases, including acne, seborrheic dermatitis, viral skin diseases, urticaria, pruritus, viral infectious diseases and contact dermatitis.

The fourth object of the present invention may be accomplished by applying an effective amount of a PPAR-α activator represented by the aforementioned formula 1 to the skin of patients suffering from skin diseases, including acne, seborrheic dermatitis, viral skin diseases, urticaria, pruritus, viral infectious diseases and contact dermatitis.

The molecules represented by the formula 1 according to the present invention acts as an effective activator of PPAR-α to increase the expression of PPAR-α in the skin, thus enabling various physiological effects of PPAR-α to be effectively used. Specifically, the inventive composition for treating skin diseases comprising the PPAR-α activator as an active ingredient is useful as an agent for treating skin diseases involving inflammatory reactions, including acne, seborrheic dermatitis, viral skin diseases, urticaria, pruritus, viral infectious diseases and contact dermatitis.

The PPAR-α activator according to the present invention is a compound represented by the following formula 1:

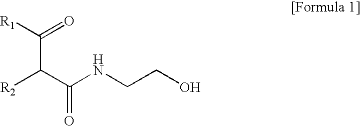

wherein R₁ and R₂ are the same or different and each represents a linear or branched C₁₋₂₂ alkyl group, a phenyl group or a benzene group.

The compound represented by the formula 1 in the present invention is preferably N-ethanol-2-myristyl or palmityl-3-oxo-stearamide or arachidamide.

The compound represented by the formula 1 in the present invention is more preferably a compound represented by the following formula 2, which is hereinafter referred to as “PC-9S”:

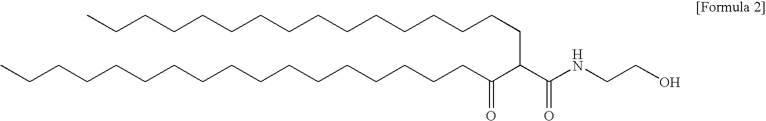

The present inventors have studied the underlying mechanisms for the therapeutic activity of topical preparations containing the compound represented by the formula 1 alleviate acne, seborrheic dermatitis, viral skin diseases, urticaria, pruritus, viral infectious diseases and contact dermatitis. As a result, the present inventors have found that the compound represented by formula 1 has an significant effects of activating PPARs, particularly PPAR-α, and shows significant anti-inflammatory effects through the aforementioned PPAR-α-activating effect. On the basis of this fact, the present invention has been completed.

The PPAR-α activator according to the present invention increases the expression of PPAR-α in the skin to enhance various physiological effects of PPAR-α, that is, the anti-inflammatory effect caused by inhibiting the activity of transcription factors such as the nuclear factor κK (NF-κK) that induces inflammatory reactions, the effect of improving the epidermal permeability barrier function, and the effect of promoting the terminal differentiation of epidermal keratinocytes.

The PPAR-α activator according to the present invention and a composition for treating skin diseases comprising the same can be effectively used to alleviate, ameliorate and treat skin diseases such as acne, seborrheic dermatitis, viral skin diseases, urticaria, pruritus, viral infectious diseases and contact dermatitis.

The content of the PPAR-α activator in the composition for treating skin diseases according to the present invention is not specifically limited, but may be about 0.05-15.0 wt %, preferably about 0.1-5.0 wt %, and more preferably about 0.3-2.5 wt %, based on the total weight of the composition. If the content of the PPAR-α activator is less than 0.05 wt %, the effect thereof will be insignificant, and if the content exceeds 15 wt %, it will not show a further increase in the effect thereof according to the increase in the amount thereof added and will lead to an increase in the production cost of the composition.

The PPAR-α activator according to the present invention and the composition for treating skin diseases comprising the same may be applied in all topical preparations for application to the skin. More specifically, the PPAR-α activator may be formulated in the form of toner, lotion, cream, essence, pack, powder, ointment, suspension, emulsion, spray, cosmetic solution, soap, shampoo, skin patch, gel, and so on. In addition, the PPAR-α activator may be formulated in the form of skin contact materials such as a cosmetic product, a detergent and a fiber.

### BRIEF DESCRIPTION OF THE DRAWINGS

The above and other objects, features and advantages of the present invention will be more clearly understood from the following detailed description taken in conjunction with the accompanying drawings, in which:

FIGS. 1 and 2 are each a graphic diagram showing the anti-inflammatory effect of a compound as a PPAR-α activator according to the present invention in a TPA-induced inflammation model;

FIGS. 3 and 4 are each a graphic diagram showing the recovery of damaged skin in atopic model mice by a compound as a PPAR-α activator according to the present invention;

FIG. 5 is a graphic diagram showing the alleviation of inflammation in atopic model mice by a compound as a PPAR-α activator according to the present invention;

FIG. 6 is a graphic diagram showing the activation of antimicrobial peptides in atopic model mice by a compound as a PPAR-α activator according to the present invention;

FIG. 7 is a photographic view of tissue cross-section which shows an increase in the expression of CRAMP (cathelicidin related murine antimicrobial peptide) in the skin's horny layer by a compound as a PPAR-α activator according to the present invention;

FIG. 8 is a graphic diagram showing the activation of PPAR-α, β/δ in atopic model mice by a compound as a PPAR-α activator according to the present invention;

FIG. 9 is a graphic diagram showing the activation of SPT (serine palmitoyl transferase) in atopic model mice by a compound as a PPAR-α activator according to the present invention; and

FIG. 10 is a photographic view showing an increase in differentiation in the epidermis of atopic model mice by a compound as a PPAR-α activator according to the present invention.

### DETAILED DESCRIPTION OF THE INVENTION

Hereinafter, the present invention will be described in further detail with reference to examples. It is to be understood, however, that these examples are illustrative only and the scope of the present invention is not limited thereto.

### EXAMPLE 1

### Effects on the Acute Inflammation Animal Model

120 mice were divided into 6 groups, each consisting of 20 mice. Group 1 was untreated as a control, group 2 was treated only with 12-O-tetradecanoylphorbol-13-acetate (TPA) as a positive control, group 3 was treated with Physiogel™ containing physiologic lipid mixture (manufactured by “A” company) as a comparative example, and groups 4, 5 and 6 were treated with multi-lamellar emulsion (MLE), MLE containing aforementioned PC-9S (0.6%) (MLE/PC-9S 1) and MLE containing aforementioned PC-9S (1.2%) (MLE/PC-9S 2), respectively. As used herein, the term multi-lamellar emulsion (MLE) means the emulsions with multi-layered structures and expresses the “Maltese cross” structure under cross-polarized microscope.

First, mice were sensitized by topically applying TPA (1 μg/20 μl acetone) to the ear. At 6 days from the next day, 20 μl of TPA was topically applied to the same site of mouse ear. At 1 hour and 8 hours after application of TPA, 20 μl of each of Physiogel™, MLE, MLE/PC-9S 1 and MLE/PC-9S 2 were topically applied to the same site in which TPA has been applied. At day 7, each of Physiogel™, MLE, MLE/PC-9S 1 and MLE/PC-9S 2 was topically applied to the mouse ear at 1 hour after application of 20 μl TPA.

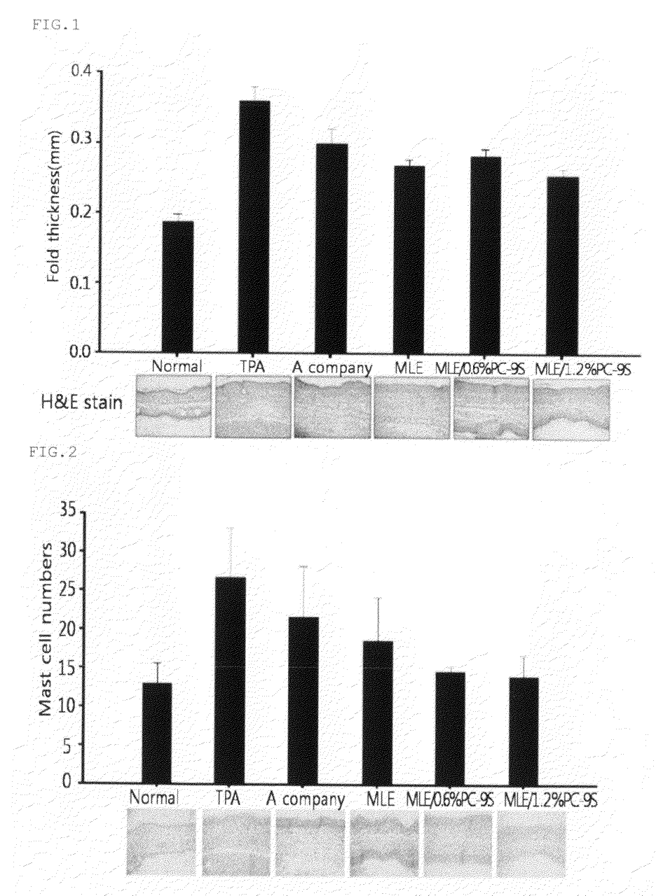

After 3 hours, the ear thickness of the mice and the number of mast cells in the mouse ear were measured, and the measurement results are shown in FIGS. 1 and 2.

As can be seen in FIGS. 1 and 2, the compound according to the present invention showed significant anti-inflammatory effects in the TPA-induced inflammation model, compared with negative control groups and Physiogel™ applied groups.

### EXAMPLE 2

### Alleviating Effects on the Atopic Dermatitis Animal Model

140 hairless mice (SKH) were divided into 7 groups, each consisting of 20 mice. Group 1 was untreated as a control, group 2 was treated with acetone, group 3 was treated with the PPAR-α activator Wy14,643, group 4 was treated with Physiogel™ containing physiologic lipid mixture (manufactured “A” company) as a comparative example, and groups 5, 6 and 7 were treated with MLE, MLE/PC-9S 1 and MLE/PC-9S 2, respectively.

100 μl of 1% oxazolone (4-ethoxymethylene-2-phenoxazol-5-one, OXA) was topically applied to both flanks of the hairless mice, and after 7 days, 100 μl of 0.1% OXA was topically applied to the same site at 2-day intervals for 10 days.

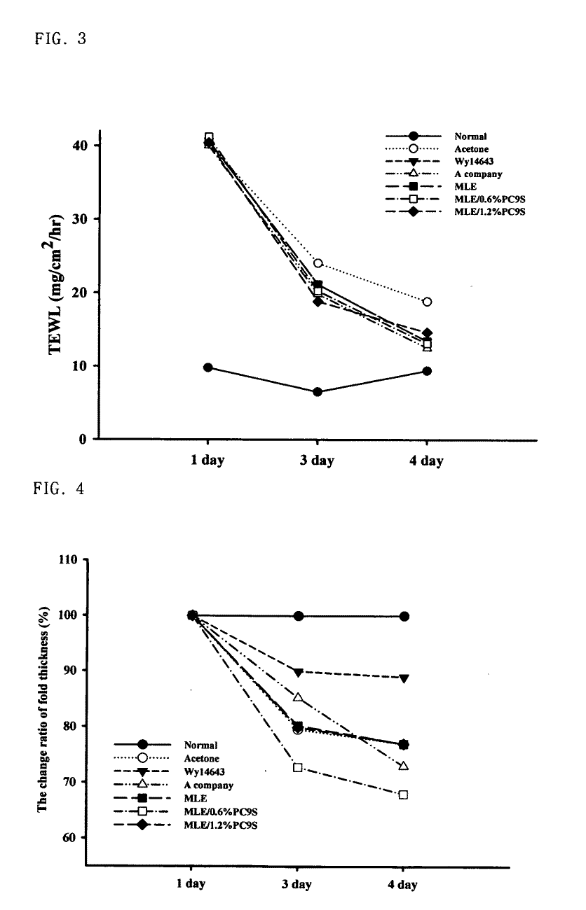

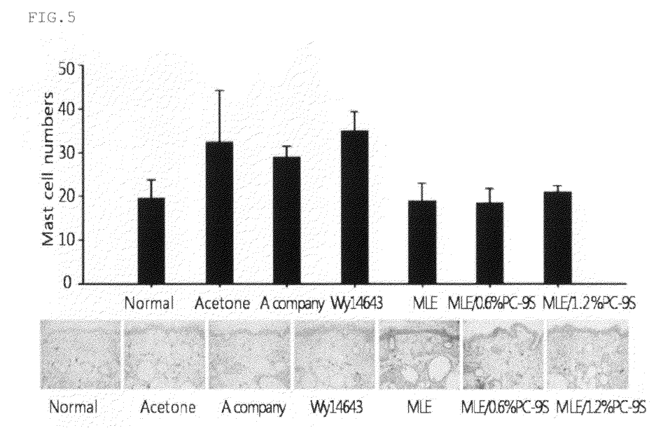

After atopic induction in the mice, each of the above-described treating agents was applied to the mouse groups twice a day for 4 days. During the application of each treating agent, the transepidermal water loss (TEWL) and fold thickness of the mouse skin were measured, and the measurement results are shown in FIGS. 3 and 4.

The above results demonstrate that the topical formulations containing the compound according to the present invention shows significant effects of restoring damaged skin barrier function. Also, it could be observed that the skin thickening by the inflammation became normalized by the treatment with the compound of the present invention.

### EXAMPLE 3

### Measurement of Epidermal Mast Cells

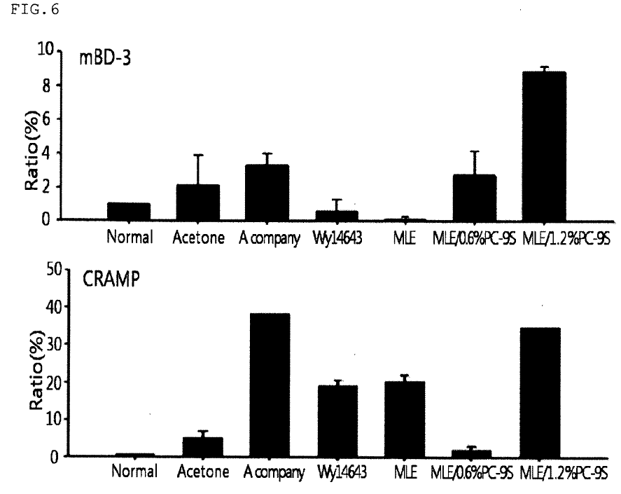

Atopic model mice induced by oxazolone (4-ethoxymethylene-2-phenoxazol-5-one, OXA) were applied with the treating agents described in Example 2, and then the number of epidermal mast cells in the mice was measured in order to measure the inflammation-alleviating effects of the treating agents. The measurement results are shown in FIG. 5.

As can be seen in FIG. 5, the compound of the present invention showed a significant inflammation-alleviating effect in the atopic model mice.

### EXAMPLE 4

### Measurement (I) of Antimicrobial Peptides

In order to examine whether the results of Example 2 induce the expression of antimicrobial peptides, the degrees of epidermal expression of mouse beta defensin-3 (mBD-3) and cathelicidin-related murine antimicrobial peptide (CRAMP) were measured.

The skin was collected from the treated sites of the atopic model mice tested in Example 2, and then total RNA was harvested from the collected skin. In order to harvest the total RNA, 1 ml of Trizol reagent was added to the collected skin. After the skin tissue was allowed to react with the Trizol reagent at room temperature for 15 seconds, 200 μl of chloroform was added thereto. Then, the tissue was centrifuged at 13,000 rpm for 10 minutes, and the supernatant was transferred into another tube. Then, 500 μl of isopropanol was added thereto, and the tissue solution was centrifuged again at 13,000 rpm for 10 minutes. The precipitated RNA was washed twice with 70% ethanol, and then diluted in triple-distilled water. The diluted RNA was quantitatively analyzed at a wavelength between 260 nm and 280 nm.

The RNA thus obtained was subjected to reverse transcriptase polymerase chain reaction (RT-PCR), in order to quantitate the messenger RNA expressions.

For RT-PCR, 2 μl MgCl₂, 1 μl RT buffer, 1 μl dNTP mix, 0.25 μl Rnase inhibitor, 0.5 μl RTase, 0.5 μl oligo dT, 3.75 μl distilled water and 2 μg RNA were placed in a tube, and then subjected to an RT-PCR reaction. The RT-PCR reaction was performed at 45° C. for 1 hour and 95° C. for 5 minutes. For the qualitative analysis of GAPDH (glyceraldehyde 3-phosphate dehydrogenase) and PPAR-α, PCR was performed using the following primers:

| | |
|---|---|
| GAPDH sense: | 5′-GGG CAT GAA CCA TGA GAA GT-3′ |
| GAPDH anti-sense: | 5′-GTC TTC TGG GTG GCA GTG AT-3′ |
| mBD-3 sense: | 5′-ATT TCT CCT GGT GCT GCT GT-3′ |
| mBD-3 anti-sense: | 5′-GGA ACT CCA CAA CTG CCA AT-3′ |
| CRAMP sense: | 5′-CGA GCT GTG GAT GAC TTC AA-3′ |
| CRAMP anti-sense: | 5′-TCC TTC ACT CGG AAC CTC AC-3′ |

For PCR, 12.5 μl PCR premix, 1 μl primer sense (10 μM), 2 μl primer anti-sense (10 μM), 1.5 μl cDNA and 7 μl distilled water were placed in a tube, and then subjected to a PCR reaction. The PCR of PPAR-α was performed in the following conditions: 30 cycles of 94° C. for 30 sec, 59° C. for 30 sec and 72° C. for 30 sec, followed by 72° C. for 10 minutes. After the PCR amplification, the resulting products were mixed with each other, and the final solution was loaded onto 1.5% agarose gel stained with a nucleic acid-specific fluorescent compound (such as, ethidium bromide) allowing visualization under UV light. The sample on the gel was visualized under UV light in a dark room and photographed with a computer-coupled camera. The gel photograph was analyzed in image processing software allowing band strength to be quantified. To further clarify the results, real-time PCR was performed. 5 μl cybergreen, 2 μl primer sense (10 μM), 2 μl primer anti-sense (10 μM), 1 μl cDNA and 1 μl distilled water were placed in a tube, and then subjected to real-time PCR.

The PCR results were analyzed using a program, and the analysis results are shown in FIG. 6.

As can be seen in FIG. 6, it was found that the compound according to the present invention significantly activated the antimicrobial peptides.

### EXAMPLE 5

### Measurement of Epidermal Antimicrobial Peptides Expressions (II)

In order to examine whether the results of Example 2 induce the expression of an antimicrobial peptide, the expression of CRAMP in the skin was measured. The skin was collected from the treated sites of the atopic model mice prepared in Example 2, and then made into a paraffin block. The tissue was attached to a slide using a paraffin microtome, and 500 μl of peroxidase blocking reagent was loaded onto the slide, and then incubated for 30 minutes. Then, the slide was washed three times with PBS buffer. 500 μl of protein blocking reagent was loaded onto the slide, and then incubated for 15 minutes. The tissue on the slide was incubated with primary goat anti-mouse CRAMP at 25° C. for 30 minutes and incubated with donkey anti-goat horse reddish peroxidase as a secondary antibody at 25° C. for 30 minutes. The tissue on the slide was incubated with the color development reagent DAB at 25° C. for 10 minutes. After completion of the incubation, the expression of CRAMP in the horny layer of the skin was measured with a microscope.

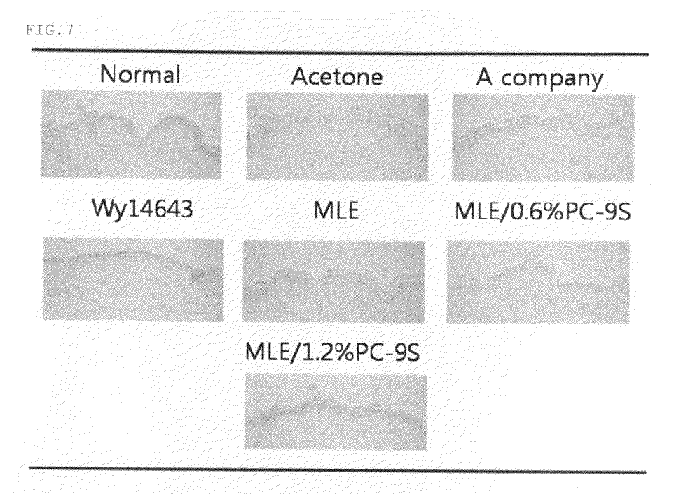

It was measured that the expression of CRAMP in the skin horny layer of the mice applied with the compound of the present invention significantly increased, and the measurement results are shown in FIG. 7.

### EXAMPLE 6

### Measurement of Activation of PPAR-α,β/δ

In order to examine whether the results of Example 2 are attributable to the epidermal activation of PPAR-α, β/δ, the activation of PPAR-α, β/δ in the skin was measured using the following primers:

| | |
|---|---|
| PPAR-α sense: | 5′-CCTCAGGGTACCACTACGGAGT-3′ |
| PPAR-α anti-sense: | 5′-GCCGAATAGTTCGCCGAA-3′ |
| PPAR-β/67 sense: | 5′-TGG AGC TCG ATG ACA GTG AC-3′ |
| PPAR-β/δ | 5′-TGT CCT GGA TGG CTT CTA CC-3′ |
| anti-sense: | |

Real-time PCR was performed. For this purpose, 5 μl cybergreen, 2 μl primer sense (10 μM), 2 μl primer anti-sense (10 μM), 1 μl cDNA and 1 μl distilled water were placed in a tube, and then subjected to real-time PCR.

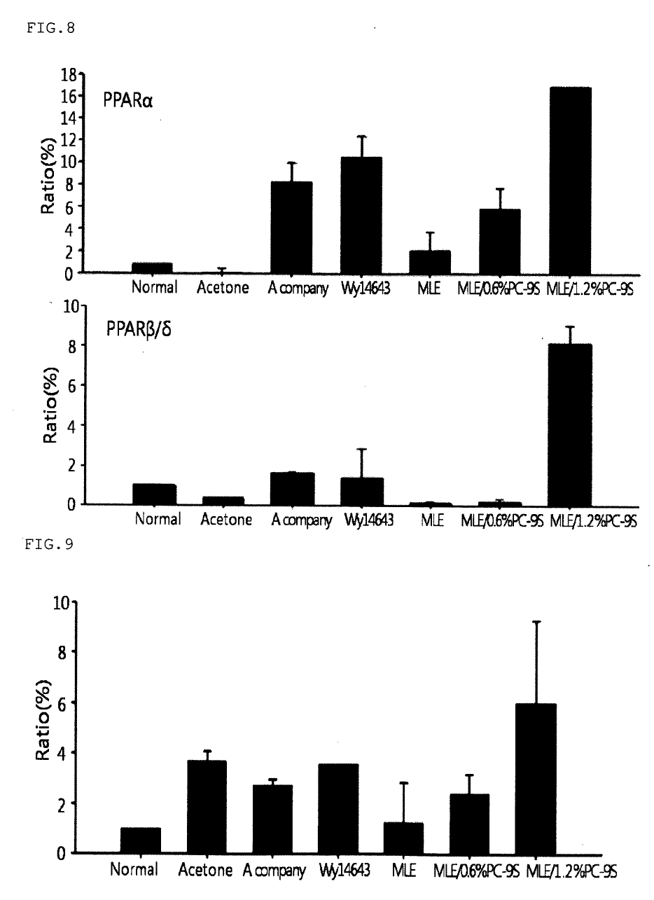

The PCR results were analyzed using a program, and the analysis results are shown in FIG. 8.

As can be seen in FIG. 8, it was found that the compound according to the present invention activated PPAR-α, β/δ.

### EXAMPLE 7

### Measurement of Activation of Serine Palmitoyl Transferase (SPT)

In order to examine whether the results of Example 2 induce the activation of SPT that promotes the in situ synthesis of ceramide, the activation of SPT in the skin was measured using the following primers:

| | |
|---|---|
| SPT sense: | 5′-GAA GAA CTA GAG AAA TTG GTA GCA |
| | AG-3′ |
| SPT anti-sense: | 5′-TTC AGC TCA TCA CTC AGA ATC |
| | AG-3′ |

Real-time PCR was performed. For this purpose, 5 μl cybergreen, 2 μl primer sense (10 μM), 2 μl primer anti-sense (10 μM), 1 μl cDNA and 1 μl distilled water were placed in a tube, and then subjected to a PCR reaction.

The PCR results were analyzed using a program, and the analysis results are shown in FIG. 9.

The obtained results revealed that the compound according to the present invention significantly activates SPT.

### EXAMPLE 8

### Measurement of Terminal Differentiation of Epidermal Keratinocytes

In order to examine whether the results of Example 2 induce the terminal differentiation of the epidermal keratinocytes, keratinocyte differentiation in the skin was measured. The skin was collected from the treated sites of the atopic model mice prepared in Example 2, and then made into a paraffin block. The skin tissue was attached to a slide using a paraffin microtome, and 500 μl of peroxidase blocking reagent was loaded onto the slide, and then incubated for 30 minutes. Then, the slide was washed three times with PBS buffer at a 5-minute interval. 500 μl of protein blocking reagent was loaded onto the slide, and then incubated for 15 minutes. The tissue on the slide was incubated with primary anti-mouse filaggin, loricrin and involucrin at 25° C. for 30 minutes and incubated with donkey anti-rabbit HRP as a secondary antibody at 25° C. for 30 minutes. The tissue on the slide was incubated with the color development reagent DAB at 25° C. for 10 minutes. After completion of the incubation, the expression of each of the differentiation markers in the skin horny layer was measured with a microscope.

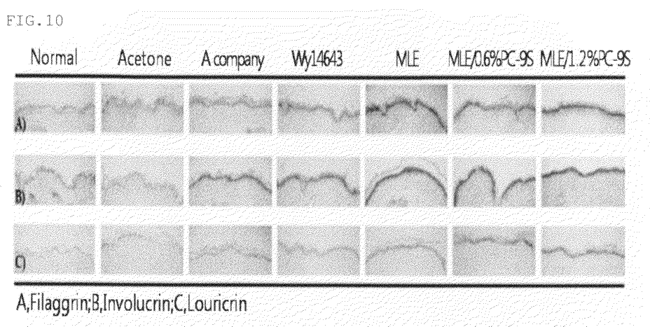

As a result, it was measured that differentiation in the skin horny layer of the mice applied with the pseudo-ceramide compound of the present invention increased, and the measurement results are shown in FIG. 10.

### EXAMPLE 9

### Evaluation of Safety Against Skin Irritation

In order to evaluate the safety of the PPAR-α activator compound according to the present invention for application as a skin external preparation, an animal toxicity test and a human patch test were performed.

To perform this evaluation, a single dose oral toxicity test using rats, a skin irritation test using rabbits, a skin sensitization test using guinea pigs and an ophthalmic mucous membrane irritation test using rabbits were performed as toxicity tests for PC-9S in animals. Those tests were performed based on “Toxicity Test Standards for Pharmaceutical Products” disclosed by the Korea Food & Drug Administration. Additionally, a human patch test for PC-9S was carried out on 30 subjects (average age: 24.1). As a result, as shown in Table 1 below, a slight skin irritation was observed only in the skin irritation test using rabbits. However, when evaluating the overall results obtained from the other toxicity tests and the human patch test, it was considered that the pseudo-ceramide compound would not pose a safety problem.

TABLE 1
Evaluation of skin irritation safety

| Test Items | Results |
|---|---|
| Single dose oral toxicity test (toxicity test) | No irritation |
| Skin irritation test using rabbits | Slight irritation |
| Skin sensitization test using guinea pigs | No irritation |
| Ophthalmic mucous membrane irritation test using | No irritation |
| rabbits | |
| Human patch test (1% PC-9S) | No irritation |
| Human patch test (10% PC-9S) | No irritation |

Formulation 1: Emollient Cream

A moisturizing agent was added to purified water and heated to 70° C. PC-9S and oil phase components were dissolved by heating, and an emulsifier, a preservative, or the like were added thereto, followed by heating to 70° C. The oil phase was added to the above aqueous phase. Then, the emulsified particles were homogenized with a homomixer, followed by debubbling, filtration and cooling.

TABLE 2

| Function | Components | Content (%) |
|---|---|---|
| Active ingredient | PC-9S | 1.2 |
| Oil phase components | Cetostearyl alcohol | 6.0 |
| | Stearic acid | 2.0 |
| | Lanolin | 3.0 |
| | Squalane | 6.0 |
| | Octyldodecanol | 9.0 |
| Moisturizing agent | 1,3-butylene glycol | 4.0 |
| | Glycerin | 2.0 |
| Emulsifier | POE(25) cetyl alcohol ether | 4.0 |
| | Glycerin monostearate | 2.5 |
| Preservative | Methyl paraben | q.s. |
| | Purified Water | balance |

Formulation 2: Ointment for External Use

PC-PS and oil phase components were dissolved by heating, and an emulsifier, a preservative, or the like was added thereto, followed by adjustment of the temperature to 70° C. The resultant mixture was mixed homogeneously in a homomixer, followed by debubbling, filtration and cooling.

TABLE 3

| Function | Components | Content (%) |
|---|---|---|
| Active ingredient | PC-9S | 0.6 |
| Oil phase components | Petrolatum | balance |
| | Cetostearyl alcohol | 2.5 |
| | Lanolin | 3.5 |
| | Squalane | 3.5 |
| Emulsifier | Ceteareth-20 | 4.0 |
| Preservative | Propyl paraben | q.s. |
| | Methyl paraben | q.s. |

Although the preferred embodiment of the present invention has been described for illustrative purposes, those skilled in the art will appreciate that various modifications, additions and substitutions are possible, without departing from the scope and spirit of the invention as disclosed in the accompanying claims.

1. A skin disease treating agent comprising an effective amount of a peroxisome proliferator activated receptor-α (PPAR-α) activator represented by the following Formula 1, as an agent for treating skin diseases involving inflammatory reactions, including acne, seborrheic dermatitis, viral skin diseases, urticaria, pruritus, viral infectious diseases and contact dermatitis: 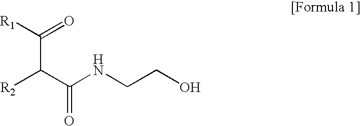
wherein R₁ and R₂ are the same or different and each is a member selected from the group consisting of a linear or branched C₁₋₂₂ alkyl group, a phenyl group and a benzene group.

2. The skin disease treating agent according to claim 1, wherein R₁ and R₂ in Formula 1 each has 16 to 18 carbon atoms.

3. The skin disease treating agent according to claim 1, wherein the PPAR-α activator represented by Formula 1 comprises a member selected from the group consisting of N-ethanol-2-myristyl, palmityl-3-oxo-stearamide and arachidamide.

4. The skin disease treating agent according to claim 1, wherein the PPAR-α activator represented by Formula 1 is a ceramide compound represented by the following Formula 2:

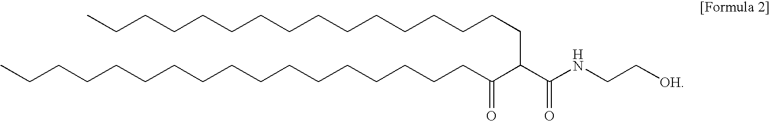

5. A method of using a peroxisome proliferator activated receptor-α (PPAR-α) activator according to claim 1 to prepare a composition for treating skin diseases involving inflammatory reactions, including acne, seborrheic dermatitis, viral skin diseases, urticaria, pruritus, viral infectious diseases and contact dermatitis, wherein the PPAR-α activator is being used in an amount of 0.05-15.0 wt % based on the total weight of the composition.

6. A method for activating PPAR-α, comprising: applying an effective amount of a peroxisome proliferator activated receptor-α (PPAR-α) activator according to claim 1 to the skin of patients suffering from skin diseases involving inflammatory reactions, including acne, seborrheic dermatitis, viral skin diseases, urticaria, pruritus, viral infectious diseases and contact dermatitis.

7. A method for treating skin diseases, comprising: applying an effective amount of a peroxisome proliferator activated receptor-α (PPAR-α) activator according to claim 1 to the skin of patients suffering from skin diseases involving inflammatory reactions, including acne, seborrheic dermatitis, viral skin diseases, urticaria, pruritus, viral infectious diseases and contact dermatitis.

8. A method of using a peroxisome proliferator activated receptor-α (PPAR-α) activator according to claim 2 to prepare a composition for treating skin diseases involving inflammatory reactions, including acne, seborrheic dermatitis, viral skin diseases, urticaria, pruritus, viral infectious diseases and contact dermatitis, wherein the PPAR-α activator is being used in an amount of 0.05-15.0 wt % based on the total weight of the composition.

9. A method of using a peroxisome proliferator activated receptor-α (PPAR-α) activator according to claim 3 to prepare a composition for treating skin diseases involving inflammatory reactions, including acne, seborrheic dermatitis, viral skin diseases, urticaria, pruritus, viral infectious diseases and contact dermatitis, wherein the PPAR-α activator is being used in an amount of 0.05-15.0 wt % based on the total weight of the composition.

10. A method of using a peroxisome proliferator activated receptor-α (PPAR-α) activator according to claim 4 to prepare a composition for treating skin diseases involving inflammatory reactions, including acne, seborrheic dermatitis, viral skin diseases, urticaria, pruritus, viral infectious diseases and contact dermatitis, wherein the PPAR-α activator is being used in an amount of 0.05-15.0 wt % based on the total weight of the composition.

11. A method for activating PPAR-α, comprising:
applying an effective amount of a peroxisome proliferator activated receptor-α (PPAR-α) activator according to claim 2 to the skin of patients suffering from skin diseases involving inflammatory reactions, including acne, seborrheic dermatitis, viral skin diseases, urticaria, pruritus, viral infectious diseases and contact dermatitis.

12. A method for activating PPAR-α, comprising:
applying an effective amount of a peroxisome proliferator activated receptor-α (PPAR-α) activator according to claim 3 to the skin of patients suffering from skin diseases involving inflammatory reactions, including acne, seborrheic dermatitis, viral skin diseases, urticaria, pruritus, viral infectious diseases and contact dermatitis.

13. A method for activating PPAR-α, comprising:
applying an effective amount of a peroxisome proliferator activated receptor-α (PPAR-α) activator according to claim 4 to the skin of patients suffering from skin diseases involving inflammatory reactions, including acne, seborrheic dermatitis, viral skin diseases, urticaria, pruritus, viral infectious diseases and contact dermatitis.

14. A method for treating skin diseases, comprising:
applying an effective amount of a peroxisome proliferator activated receptor-α (PPAR-α) activator according to claim 2 to the skin of patients suffering from skin diseases involving inflammatory reactions, including acne, seborrheic dermatitis, viral skin diseases, urticaria, pruritus, viral infectious diseases and contact dermatitis.

15. A method for treating skin diseases, comprising:
applying an effective amount of a peroxisome proliferator activated receptor-α (PPAR-α) activator according to claim 3 to the skin of patients suffering from skin diseases involving inflammatory reactions, including acne, seborrheic dermatitis, viral skin diseases, urticaria, pruritus, viral infectious diseases and contact dermatitis.

16. A method for treating skin diseases, comprising:
applying an effective amount of a peroxisome proliferator activated receptor-α (PPAR-α) activator according to claim 4 to the skin of patients suffering from skin diseases involving inflammatory reactions, including acne, seborrheic dermatitis, viral skin diseases, urticaria, pruritus, viral infectious diseases and contact dermatitis.
